(19) United States
(12) Patent Application Publication
(10) Pub. No.: US 2023/0330326 A1
(43) Pub. Date: Oct. 19, 2023

(54) ELECTROOSMOTIC PUMP, INSULIN PUMP AND INSULIN PUMP SYSTEM

(71) Applicant: Peking University, Haidian District (CN)

(72) Inventor: Yue CUI, Haidian District (CN)

(73) Assignee: Peking University, Haidian District (CN)

(21) Appl. No.: 18/096,061

(22) Filed: Jan. 12, 2023

(30) Foreign Application Priority Data
Apr. 15, 2022 (CN) 202210393564.0
Apr. 15, 2022 (CN) 202210394009.X

(51) Int. Cl. A61M 5/142; B01D 61/42; A61B 5/145

(52) U.S. Cl. CPC A61M 5/14244; B01D 61/427; A61B 5/14532

(57) ABSTRACT

The present disclosure relates to the technical field of electroosmotic pumps (EOPs) and wearable medical equipment, and in particular, to an EOP, an insulin pump and an insulin pump system. The EOP of the present disclosure is cost-effective and has low power consumption, and can replace an existing mechanical pump device as a driving device in the insulin pump to achieve miniaturization of the insulin pump and lower the price of the insulin pump. In addition, when the EOP adopts a modified flexible membrane with penetrating pores, the EOP can pump and infuse a high-concentration insulin solution to achieve miniaturization and high efficiency of the insulin pump, which is conducive to the integration of wearable medical devices for treating diabetes.

### CROSS REFERENCE TO RELATED APPLICATION

This application claims priority to the Chinese Patent Application No. 202210393564.0, filed with China National Intellectual Property Administration (CNIPA) on Apr. 15, 2022, and entitled “ELECTROOSMOTIC PUMP (EOP) AND INSULIN PUMP SYSTEM” and the Chinese Patent Application No. 202210394009.X, filed with CNIPA on Apr. 15, 2022, and entitled “ELECTROOSMOTIC PUMP (EOP) AND APPLICATION THEREOF”, which are incorporated herein by reference in their entireties.

### TECHNICAL FIELD

The present disclosure relates to the technical field of electroosmotic pumps (EOPs) and wearable medical equipment, and in particular, to an EOP, an insulin pump and an insulin pump system.

### BACKGROUND

With the continuous improvement of real living standards, the number of diabetic patients caused by high sugar intake is rising. Insulin injection, as a routine treatment for diabetes, is used by more and more people. The existing insulin injection methods include manual injection before meals and automatic injection using an insulin pump. The use of insulin pump for insulin infusion has the advantages of fine, stable, and flexible administration, and patients no longer receive insulin subcutaneously through a syringe or injection pen. The insulin pump has a relatively excellent diabetes treatment effect due to its preciseness.

The insulin pump usually consists of a pump device, an insulin storage device (a small syringe), and an infusion device connected thereto. After the syringe is loaded into the pump, the insulin storage device punctures the guide needle at the front end of the connected infusion tube into the patient's skin (usually abdominal wall) with a needle injector, and the piston of the small syringe is pushed by the screw motor of the electric insulin pump to infuse the insulin into the body. The basic purpose of the insulin pump is to simulate the secretory function of the pancreas, and continuously inject insulin into the user's subcutaneous according to the dosage required by the human body, so as to maintain the stability of blood glucose throughout the day, thereby controlling diabetes.

However, the existing insulin pumps are expensive. The imported pumps cost ranging from 40,000 to 50,000 RMB each (around 7,000 USD), and the domestic pumps cost ranging from 20,000 to 30,000 RMB each (around 4,000 USD). In addition, the existing insulin pumps are usually small with a fixed shape, which is difficult to further achieve miniaturization and flexibility. Therefore, it is required to develop a cost-effective miniaturized insulin pump with a low power consumption, high efficacy, and excellent flexibility.

### SUMMARY

An objective of the present disclosure is to provide an EOP, an insulin pump and an insulin pump system. The EOP of the present disclosure is cost-effective and has low power consumption, and can replace an existing mechanical pump device as a driving device in the insulin pump to achieve miniaturization of the insulin pump and lower the price of the insulin pump. In addition, when the EOP adopts a modified flexible membrane with penetrating pores, the EOP can pump and infuse a high-concentration insulin solution to realize miniaturization and high efficiency of the insulin pump, which is conducive to integrating wearable medical equipment for treating diabetes.

To achieve the above objective of the present disclosure, the present disclosure provides the following technical solutions.

The present disclosure provides an EOP, including a first electrode, a porous membrane, and a second electrode. The first electrode is close to one side of the porous membrane, and the second electrode is close to another side of the porous membrane.

The porous membrane is a flexible membrane with penetrating pores, or is obtained by modification of the flexible membrane with penetrating pores. The flexible membrane with penetrating pores is a polycarbonate (PC) track-etched membrane with penetrating pores, a polyester track-etched membrane with penetrating pores, a polytetrafluoroethylene track-etched membrane with penetrating pores, or a polyimide track-etched membrane with penetrating pores.

The modification includes: modifying the flexible membrane with penetrating pores by polydopamine (PDA), polyethylene glycol diamine (NH₂-PEG-NH₂), and bovine serum albumin (BSA) sequentially.

The first electrode is an anode, and is made of a material comprising a stainless steel mesh, a metal mesh, a metal coating, or a gold-plated stainless steel mesh.

The second electrode is a cathode, and is made of a material comprising a stainless steel mesh, a metal mesh, a metal coating, or a gold-plated stainless steel mesh.

Preferably, the porous membrane has a thickness of 5-200 μm.

Preferably, the flexible membrane with penetrating pores is prepared by bombarding a flexible membrane with neutrons, protons, heavy ions, or high-energy particle flows and then conducting chemical etching, wherein the flexible membrane that is bombarded is a PC membrane, a polyester membrane, a polytetrafluoroethylene membrane, or a polyimide membrane.

Preferably, the penetrating pores have a density of 10⁴-10¹⁰ pore/cm² and a pore size of 50-300 nm.

Preferably, the modification includes: soaking the flexible membrane with penetrating pores in a PDA solution with a pH of 8.5 for 3-4 h, and rinsing the soaked flexible membrane with deionized water to obtain an intermediate material; and soaking the intermediate material in a polyethylene glycol diamine solution with a pH of 8.5 for 24 h, and transferring the soaked intermediate material to a bovine serum albumin solution with a pH of 7.4 for incubation for 24 h.

Preferably, the stainless steel mesh comprises a 304 stainless steel mesh or a 316 stainless steel mesh.

The metal mesh comprises an aluminum mesh, titanium mesh, or a platinum mesh.

The metal coating comprises a gold coating or a platinum coating.

Preferably, the EOP has a thickness of 35 μm to 1.8 mm.

The present disclosure further provides an insulin pump. The EOP according to the above technical solutions is used as a driving pump to drive automatic injection of insulin.

Preferably, the insulin pump includes an insulin storage device, a power supply device, and the EOP.

The insulin storage device is configured to store insulin to be injected.

The power supply device is configured to power the EOP.

Preferably, when the porous membrane is the flexible membrane with penetrating pores, the EOP has a driving voltage of 0.1-20 V, and insulin has a concentration of 1-50 U/mL.

When the porous membrane is obtained by the modification of the flexible membrane with penetrating pores, insulin has a concentration of 1-500 U/mL.

The present disclosure further provides an insulin pump system, including an insulin pump, a sensor device, and an infusion device. The insulin pump system is configured for automatic pumping of insulin.

The insulin pump is integrated with the sensor device to form a closed-loop system, and the sensor device is configured to measure a glucose concentration, obtain a glucose detection signal, and control opening and closing of an EOP according to the detection signal.

The EOP is configured to drive insulin to flow to the infusion device.

The infusion device is configured to complete injection of insulin.

The insulin pump is the insulin pump according to the above technical solutions.

The present disclosure provides an EOP, including a first electrode, a porous membrane, and a second electrode. The first electrode is close to one side of the porous membrane, and the second electrode is close to the other side of the porous membrane. The porous membrane is a flexible membrane with penetrating pores, or is obtained by modification of the flexible membrane with penetrating pores. The flexible membrane with penetrating pores is a PC track-etched membrane with penetrating pores, a polyester track-etched membrane with penetrating pores, a polytetrafluoroethylene track-etched membrane with penetrating pores, or a polyimide track-etched membrane with penetrating pores. The modification includes: modifying the flexible membrane with penetrating pores by PDA, polyethylene glycol diamine, and BSA sequentially. The first electrode is an anode, and is made of a material comprising a stainless steel mesh, a metal mesh, a metal coating, or a gold-plated stainless steel mesh. The second electrode is a cathode, and is made of a material comprising a stainless steel mesh, a metal mesh, a metal coating, or a gold-plated stainless steel mesh.

In order to achieve the flexibility and miniaturization of the insulin pump and reduce the preparation cost of the insulin pump, the technical concept provided by the present disclosure is as follows: the PC track-etched membrane with penetrating pores, the polyester track-etched membrane with penetrating pores, the polytetrafluoroethylene track-etched membrane with penetrating pores, or the polyimide track-etched membrane with penetrating pores is used as the porous membrane to prepare the EOP, and the EOP is applied to the insulin pump to drive the continuous infusion of insulin. The PC membrane is an ideal material for preparation of the EOP due to its low cost and excellent flexibility and stability.

In addition, in order to obtain a driving pump device capable of pumping a relatively high concentration (50-100 U/mL) of insulin, and to make the EOP flexible and miniaturized, the technical concept provided by the present disclosure is as follows: the flexible membrane with penetrating pores (PC membrane, polyester track-etched membrane, polytetrafluoroethylene membrane, and polyimide membrane) is modified by PDA, polyethylene glycol diamine, and BSA sequentially. After the flexible membrane with penetrating pores is modified by the PDA, a large number of binding sites that can bind to polyethylene glycol diamine and BSA will be obtained on the surface of the membrane. These binding sites bind to polyethylene glycol diamine and the BSA in the subsequent modification process, and finally a modified flexible membrane with penetrating pores is obtained. When the membrane is used as an EOP porous membrane, polyethylene glycol diamine and BSA on the surface of the membrane can effectively reduce the adsorption of protein substances from the transported liquid to the penetrating pores and the surface of the porous membrane. Therefore, it is helpful for the EOP to pump relatively high concentrations of insulin in a stable manner. In addition, the flexible membrane material used in the present disclosure is cost-effective, has excellent flexibility and stability, and the modification process is easy to operate, which is conducive to the integration and commercialization of wearable medical devices.

Compared with the conventional insulin pump that uses a mechanical pump device to drive insulin infusion, the EOP provided by the present disclosure is used as a component (driving pump) of the insulin pump to drive insulin infusion, which adopts an unmodified or modified flexible membrane with penetrating pores as the porous membrane to reduce the volume of the EOP. In addition, when the modified flexible membrane with penetrating pores is used as the porous membrane, high-concentration insulin can also be pumped with high insulin pumping stability. In addition, the EOP provided by the present disclosure has a thickness of only 35 μm to 1.8 mm (the thickness of the porous membrane is in the micron level, and therefore, the thickness of the EOP depends on the thickness of the first electrode and the second electrode) and power consumption of only about 1 mW, costs only about 1 USD, and has a simple preparation structure, noiseless use, and low power consumption. In addition, the EOP provided by the present disclosure is soft and flexible, has high biocompatibility, is safe and sterile, and can be easily integrated into a flexible closed-loop system.

### BRIEF DESCRIPTION OF THE DRAWINGS

FIG. 1 is a schematic diagram of a cross-sectional structure of an EOP according to the present disclosure;

FIG. 2 is a structural exploded view of the EOP according to the present disclosure;

FIG. 3 is a scanning electron microscope (SEM) image of an unmodified flexible membrane with penetrating pores of the present disclosure as a porous membrane;

FIG. 4 is a diagram showing the flow rate changes of insulin driven by the EOP according to Example 1 of the present disclosure at different working voltages;

FIG. 5 is a diagram showing the current changes generated by the EOP according to Example 1 of the present disclosure at different working voltages;

FIG. 6 is a diagram showing the power consumptions for the EOP according to Example 1 of the present disclosure at different voltages;

FIG. 7 is a diagram showing the volumetric flow rates of insulin pumped by an EOP according to Example 2 of the present disclosure at different insulin concentrations; and

FIG. 8 is a diagram showing relative flow rate changes of insulin pumped by an EOP according to Example 3 of the present disclosure.

### DETAILED DESCRIPTION OF THE EMBODIMENTS

The present disclosure provides an EOP and an application thereof, and the specific implementation contents are as follows.

The present disclosure provides an EOP. The EOP includes a first electrode, a second electrode, and a porous membrane. The first electrode is an anode, and the anode is made of a material comprising a stainless steel mesh, a metal mesh, a metal coating, or a gold-plated stainless steel mesh. The second electrode is a cathode, and the cathode is made of a material comprising a stainless steel mesh, a metal mesh, a metal coating, or a gold-plated stainless steel mesh.

The EOP adopts a PC track-etched membrane with penetrating pores, a polyester track-etched membrane with penetrating pores, a polytetrafluoroethylene track-etched membrane with penetrating pores, or a polyimide track-etched membrane with penetrating pores as the porous membrane.

Or, the porous membrane is obtained by modification of the flexible membrane with penetrating pores. The modification includes: modifying the flexible membrane with penetrating pores by PDA, polyethylene glycol diamine, and BSA sequentially. The flexible membrane with penetrating pores is a PC track-etched membrane, a polyester track-etched membrane, a polytetrafluoroethylene track-etched membrane, or a polyimide track-etched membrane.

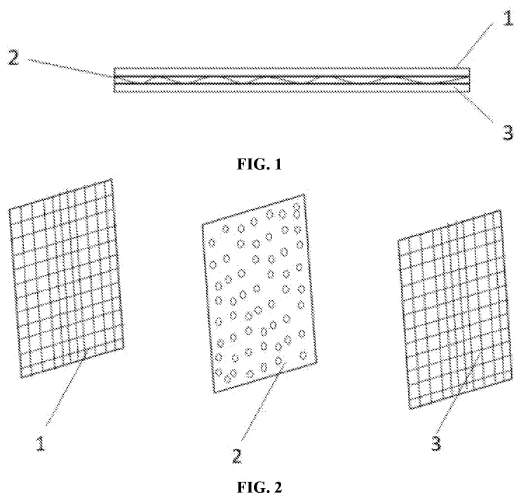

FIG. 1 is a schematic diagram of a cross-sectional structure of an EOP according to an example of the present disclosure. As shown in FIG. 1, the EOP includes a first electrode 1, a porous membrane 2, and a second electrode 3. The first electrode 1 is close to one side of the porous membrane 2, and the second electrode 3 is close to the other side of the porous membrane 2. The porous membrane 2 of the EOP can be rectangular, circular, or of any other shape. The shapes of the first electrode 1 and the second electrode 3 are adapted to the shape of the porous membrane 2.

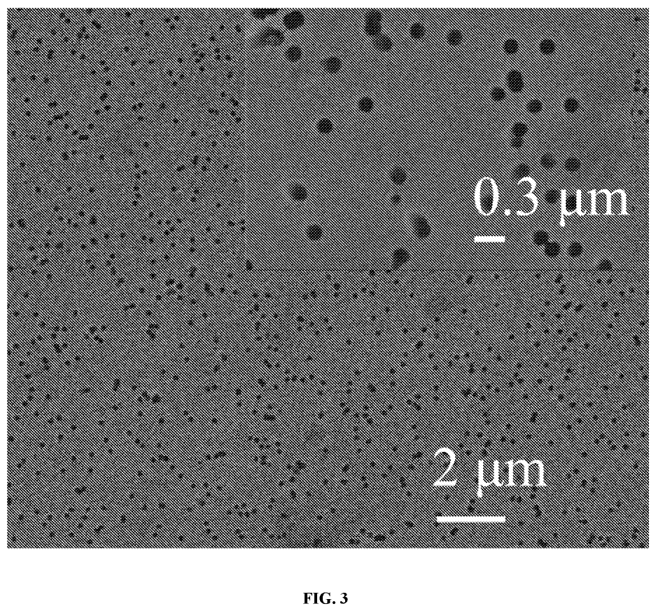

FIG. 2 is a structural exploded view of the EOP provided by the example of the present disclosure. The porous membrane shown in FIG. 2 is rectangular, and the first electrode and the second electrode are also rectangular to be adapted to the shape of the porous membrane. In addition, the material of the first electrode and the second electrode requires excellent electrical conductivity, which can be a stainless steel mesh such as a 304 stainless steel mesh and a 316 stainless steel mesh, a metal mesh such as an aluminum mesh, a titanium mesh, and a platinum mesh, can be a metal coating such as a gold metal coating and a platinum metal coating, or can be a gold-plated stainless steel mesh. In the embodiment of the present disclosure, the first electrode and the second electrode apply an electric field to the porous membrane after being energized. The inner wall of the penetrating pore of the modified poly-flexible membrane or the unmodified flexible membrane with penetrating pores (such as the PC membrane) forms an electric double layer. Under the action of the electric field, charges in the electric double layer will be driven towards the oppositely charged electrodes, and drag the surrounding liquid to flow, providing continuous infusion without mechanical wear and effectively reducing power consumption.

In a specific implementation, the present disclosure adopts the flexible PC membrane with penetrating pores, the polyester membrane with penetrating pores, the polytetrafluoroethylene membrane with penetrating pores, or the polyimide membrane with penetrating pores as the raw material for preparation of the porous membrane in the EOP, and chemical etching is conducted to form the porous membrane. Both sides of the porous membrane are supplemented with the first electrode and the second electrode capable of applying an electric field to the porous membrane.

In a specific implementation, the present disclosure can also adopt the flexible membrane material with penetrating pores as the raw material for preparation of the porous membrane in the EOP, such as the PC track-etched membrane, the polyester track-etched membrane, the polytetrafluoroethylene track-etched membrane, and the polyimide track-etched membrane. These flexible membranes with penetrating pores are modified by PDA, polyethylene glycol diamine, and BSA sequentially, so as to be used as the porous membrane. Both sides of the porous membrane are supplemented with the first electrode (anode) and the second electrode (cathode) capable of applying an electric field to the porous membrane. The first electrode is close to one side of the porous membrane, and the second electrode is close to the other side of the porous membrane.

In some embodiments, a method for preparing the porous membrane includes: soaking the flexible membrane with penetrating pores in a PDA solution with a pH of 8.5 for 3-4 h, and rinsing the soaked flexible membrane with deionized water to obtain an intermediate material; and soaking the intermediate material in a polyethylene glycol diamine solution with a pH of 8.5 for 24 h, and transferring the soaked intermediate material to a bovine serum albumin solution with a pH of 7.4 for incubation for 24 h to obtain a modified PC membrane.

In a specific implementation, during the preparation of a porous membrane, a flexible membrane with penetrating pores is soaked in a PDA solution with a pH of 8.5 at a room temperature for 3 h. In this process, the super adhesion of PDA is used to form a layer of sticky coating of tens of nanometers on the surface of the PC membrane, providing a large number of binding sites for the binding of polyethylene glycol diamine and BSA on the surface of the PC membrane. The PDA modified membrane (intermediate material) is continuously placed in an NH₂-PEG-NH₂ solution (1× phosphate-buffered saline (PBS), and pH=8.5) and stored at 37° C. for 24 h. In this process, the binding sites on the surface of the intermediate material bind a large amount of polyethylene glycol (PEG). Further, the PDA/PEG modified membrane material is incubated in a bovine serum albumin solution (1× PBS, and pH=7.4) at the room temperature for 24 h. In this process, the remaining sites on the surface of the intermediate material that do not bind PEG bind to BSA during room-temperature incubation, and finally a PC membrane modified with PDA, PEG, and BSA is obtained. When the modified PC membrane is used in the porous membrane of the EOP, the PEG and the BSA can effectively reduce the adsorption of protein substances in the transported liquid on the penetrating pores and the surface of the porous membrane, which is helpful for the EOP to pump relatively high concentrations of insulinin a stable manner.

The EOP provided by the present disclosure is small in volume, soft and flexible, and can be of any shape, which is conducive to integrating wearable medical equipment.

In some embodiments, the penetrating pores have a density of 10⁴-10¹⁰ pore/cm² and a pore size of 50-300 nm.

In a specific implementation, the design of the pore size and pore density of the penetrating pores on the porous membrane, as well as the applied voltage determine the volumetric flow rate and flow rate of the infusion liquid. Taking the driving of insulin infusion as an example, the present disclosure controls the thickness of the PC membrane to be 5-200 μm, the pore size of the penetrating pores to be 50-300 nm, the density to be 10⁴-10¹⁰ pore/cm², and the driving voltage to be 0.1-20 V. Under the above conditions, when insulin is driven by the EOP, air bubbles will not be formed at the first electrode and the second electrode, which will cause the blockage of the penetrating pores, and can well ensure the uniform flow rate of insulin.

In some embodiments, the EOP may have a thickness of 35 μm to 1.8 mm.

In a specific implementation, since the porous membrane has a thickness of only 5-200 μm, the thickness of the EOP mainly depends on the selected thickness of the first electrode and the second electrode. In the embodiment of the present disclosure, when each of the first electrode and the second electrode are made of a metal coating (such as a gold metal coating and a platinum metal coating), the first electrode and the second electrode can have a thickness of 20-200 nm. Therefore, the EOP has a thickness of 5-200 μm. When the material of the first electrode and the second electrode is a metal mesh (such as an aluminum mesh, a titanium mesh, or a platinum mesh), a gold-plated stainless steel mesh, or a stainless steel mesh (such as a 304 stainless steel mesh and a 316 stainless steel mesh), the first electrode and the second electrode have a thickness of 30-800 μm. Therefore, the formed EOP has a thickness of about 35 μm to 1.8 mm.

In some embodiments, in order to ensure the pore size and pore density of the penetrating pores on the porous membrane, the PC track-etched membrane with penetrating pores, the polyester track-etched membrane with penetrating pores, the polytetrafluoroethylene track-etched membrane with penetrating pores, or the polyimide track-etched membrane with penetrating pores in the present disclosure can be formed by bombarding a PC membrane, polyester membrane, a polytetrafluoroethylene membrane, or a polyimide membrane with neutrons, protons, heavy ions, or high-energy particle flows and conducting chemical etching. FIG. 3 shows an SEM image of the porous membrane of the EOP provided by the embodiment of the present disclosure. The distribution of the penetrating pores on the PC membrane can be clearly seen from FIG. 3.

In addition, since the present disclosure uses the micron-scale flexible membrane material as the raw material of the porous membrane in the EOP, the EOP provided by the present disclosure has the characteristics of miniaturization and free deformation according to the usage scene, and is very suitable for integrating wearable medical equipment.

In a second aspect, the present disclosure provides an application of an EOP, which uses the EOP in the above first aspect as a driving pump to drive the automatic injection of insulin for the preparation of an insulin pump.

When the porous membrane is an unmodified flexible membrane with penetrating pores, the driving voltage of EOP can be 0.1-20 V, and the insulin concentration can be 1-50 U/mL.

When EOP adopts a modified flexible membrane with penetrating pores, the insulin concentration can be 1-500 U/mL, so as to achieve the infusion of high concentration insulin.

In a specific implementation, the insulin pump may include the EOP provided by the present disclosure, an insulin storage device, and a power supply device. The insulin storage device is configured to store insulin to be injected. The power supply device is configured to power the EOP.

In some embodiments, the insulin pump is configured for the automatic pumping of insulin.

In some embodiments, the voltage of the EOP to drive insulin infusion can be 0.1-20 V, and the concentration of the driven insulin can be 1-50 U/mL.

In a specific implementation, the soft and flexible EOP with a small volume provided by the present disclosure is integrated with the insulin storage device and the power supply device to form an insulin pump for insulin infusion, which can further reduce the volume and the preparation cost of the insulin pump.

In some embodiments, the insulin pump is integrated with the sensor device to form a closed-loop system, and the sensor device is configured to measure a glucose concentration, obtain a glucose concentration detection signal, and control the opening and closing of an EOP according to the detection signal.

The EOP is configured to drive insulin flow to the infusion device.

The infusion device is configured to complete the injection of insulin.

In a specific implementation, the present disclosure uses the EOP as the driving pump device of the insulin pump. After integration with the sensor device, the sensor device obtains a glucose concentration detection signal (corresponding to the glucose concentration) and compares it with the preset target concentration of the sensor device. When the glucose concentration exceeds the target concentration, the sensor device can issue a command to the insulin pump to start the EOP through the circuit board. The started EOP further releases insulin into subcutaneous tissue of the human body through the infusion device, and the automatic infusion of insulin is completed without manual operation.

The EOP and the use thereof, and the insulin pump provided by the present disclosure are described in detail below with reference to the examples, but these examples may not be understood as a limitation to the protection scope of the present disclosure.

### Example 1

Two gold-plated stainless steel meshes were placed on both sides of a PC membrane with penetrating pores to obtain an EOP. The penetrating pores have a pore size of about 200 nm. The membrane has a thickness of about 10 μm. The nanopores have a height of about 10 μm. Nanopores are randomly distributed on the surface, with an average of 3.2 pores per square micrometer. As shown in FIG. 1, the EOP includes a PC membrane with nanopores as a porous membrane and two gold-plated stainless steel meshes as the conductive electrodes. The inner walls of the nanopores of the PC membrane form an electric double layer. When an electric field was applied, the charges in the diffusion layer of the electric double layer are driven to the oppositely charged electrodes and dragged the surrounding liquid to flow. The method could provide continuous infusion without mechanical wear. Further, the EOP is connected to an external circuit. One end of the EOP is connected to an insulin storage device, and the other end is connected to a chitosan microneedle. An insulin solution with a concentration of 10 U/mL is added to a drug reservoir. Then, different voltages are applied to electrodes across the pump using an electrochemical workstation. Insulin discharged from the microtubule needle hole is absorbed by an adsorption paper in unit time. The weight gain of the absorbent paper is measured with a balance to obtain the flow rate.

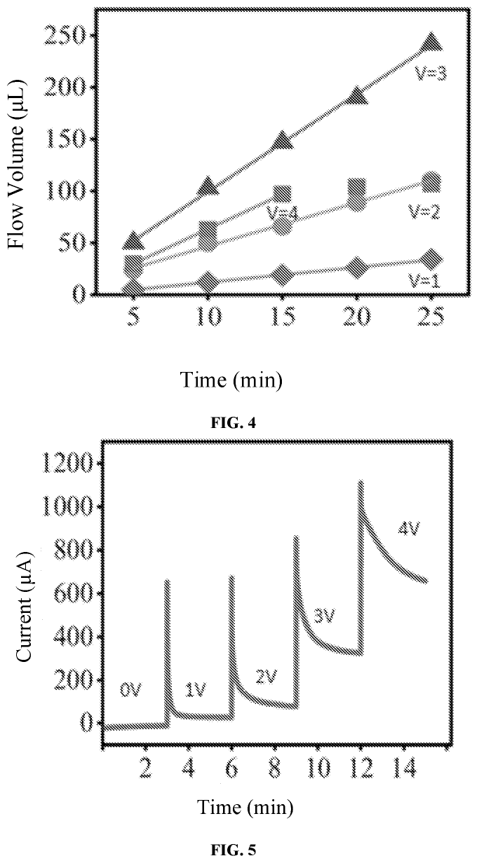

In a specific implementation, FIG. 4 is a diagram showing flow rate changes of insulin driven by the EOP provided by the example of the present disclosure under different working voltages. FIG. 4 shows a volumetric flow rate of insulin driven by the EOP under different voltages when the insulin concentration is 10 U/mL. When the voltage exceeds 3 V, air bubbles will be generated at the first electrode and the second electrode to block the penetrating pores of the porous membrane, thereby reducing the flow rate. Therefore, it is more appropriate to use 1-3 V as the working voltage of the EOP. Under 3 V, the maximum flow rate is 9.42 μL/min.

FIG. 5 is a diagram showing current changes generated by the EOP provided by the example of the present disclosure under different working voltages. As shown in FIG. 5, in the microamp range, the current increases with the applied voltage. The current range is 25-658 μA, and the voltage range is 1-4 V. A voltage of 3V generates a current of 325 μA.

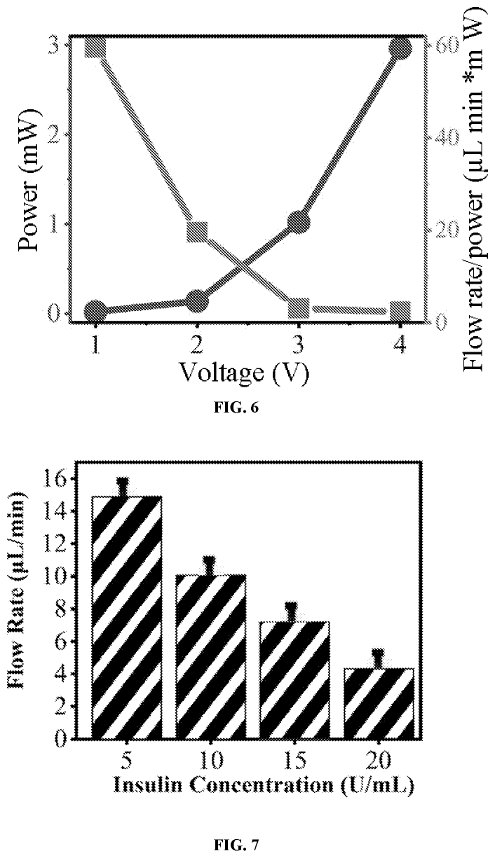

FIG. 6 is a diagram showing power consumption changes of the EOP provided by the example of the present disclosure under different voltages. As shown in FIG. 6, as the applied voltage increases from 1 V to 4 V, the power consumption of the device increases from 27 μW to 2,600 μW. After the volumetric flow rate is calculated, it is found that with the increase of voltage, the volumetric flow rate per unit power consumption decreases, and the working efficiency of the EOP is relatively low. Therefore, keeping the working voltage at 1-3 V is conducive to keeping the power consumption reasonable.

FIG. 7 is a diagram showing volumetric flow rate changes of insulin pumped by the EOP provided by the example of the present disclosure under different insulin concentrations. As shown in FIG. 7, as the insulin concentration increases from 5 U/mL to 20 U/mL, the flow rate decreases from 44 μL·min⁻¹ to 12 μL·min⁻¹, and when the insulin concentration is 50 U/mL, the liquid flow almost stops. Therefore, the EOP provided by the present disclosure, as the driving pump device for insulin, can drive insulin at a concentration of 1-50 U/mL.

### Example 2

A polyimide membrane is bombarded with high-energy particle flows and subjected to chemical etching to form a polyimide track-etched membrane with penetrating pores. Two stainless steel meshes are placed on both sides of the polyimide track-etched membrane with penetrating pores to obtain an EOP. The penetrating pores have a pore size of about 80 nm. The membrane has a thickness of 50 μm. The nanopores have a height of 50 μm. Nanopores are randomly distributed on the surface, with an average of 4 pores per square micrometer. When an electric field is applied, the charges in the diffusion layer of the electric double layer are driven to the oppositely charged electrodes and dragged the surrounding liquid to flow. The method could provide continuous infusion without mechanical wear.

The EOP prepared in this example is similar to the appearance diagram in Example 1, and the difference is only in color and size. In order to save space for the drawings in the description, this example is not repeated.

### Example 3

Preparation of a PC membrane with penetrating pores: a PC membrane is bombarded with high-energy particle flows and subjected to chemical etching to prepare the PC membrane with penetrating pores. The penetrating pores are randomly distributed.

A modification process of the PC membrane with penetrating pores is as follows: the membrane is soaked in a PDA solution (dissolved in 1× PBS with a pH of 8.5) at a room temperature for 3 h. The newly modified membrane is then rinsed thoroughly with deionized water. Then, the PDA modified membrane is placed in an NH₂-PEG-NH₂ solution (1× PBS, and pH=8.5) and stored at 37° C. for 24 h. Finally, the PDA/PEG modified membrane is incubated in a bovine serum albumin solution (1× PBS, and pH=7.4) at the room temperature for 24 h.

Preparation of an EOP: an aluminum mesh is used as anode and a 304 stainless steel mesh is used as a cathode. The modified PC membrane with penetrating pores has a width of 1.5 cm and a length of 2 cm. The anode and cathode are placed close to both sides of the modified PC membrane with penetrating pores. A voltage is applied to the anode and cathode, and the EOP could work.

In the process of measuring the flow rate, the EOP prepared in this example is used to pump insulin at a concentration of 100 U/mL. Insulin released by the micropump is collected daily. The weight of the released insulin is measured with a balance. The flow rate is calculated by converting weight to volume based on the density of each insulin injection.

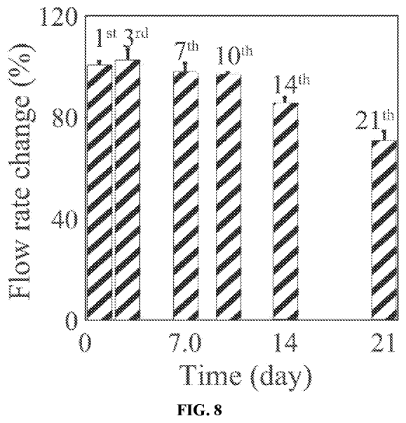

FIG. 8 is a diagram showing the relative flow rate changes of insulin pumped by an EOP provided by embodiments of the present disclosure. As shown in FIG. 8, for pumping insulin at a concentration of 100 U/mL, the flow rate of insulin pumped by the EOP provided by this embodiment is 100% on day 1, so that on day 21, a 70% release rate is still maintained relative to the flow rate on day 1. The unmodified EOP device can only release low concentrations of insulin (concentrations less than 20 U/mL) and the stability decreases over time. The overall release of an unmodified EOP device can last 1-2 days, after which the rate becomes 0.

### Example 4

Preparation of a polyester membrane with penetrating pores: a polyester membrane is bombarded with a stream of high-energy particles and chemically etched to prepare a PC film with penetrating pores. The penetrating pores are randomly distributed.

A modification process of the polyester membrane with penetrating pores is as follows: the membrane is soaked in a PDA solution (dissolved in 1× PBS with a pH of 8.5) at a room temperature for 3 h. The newly modified membrane is then rinsed thoroughly with deionized water. Then, the PDA modified membrane is placed in an NH₂-PEG-NH₂ solution (1× PBS, and pH=8.5) and stored at 37° C. for 24 h. Finally, the PDA/PEG modified membrane is incubated in a bovine serum albumin solution (1× PBS, and pH=7.4) at the room temperature for 24 h.

Preparation of an EOP: an aluminum mesh is used as anode and a 304 stainless steel mesh is used as a cathode. The modified polyester membrane with penetrating pores has a width of 2 cm and a length of 2 cm. The anode and cathode are placed close to each side of the modified polyester membrane with penetrating pores. When a voltage is applied to the anode and cathode, and the EOP can work.

The above descriptions are merely preferred implementations of the present disclosure. It should be noted that those of ordinary skill in the art may further make several improvements and modifications without departing from the principle of the present disclosure, but such improvements and modifications should be deemed as falling within the protection scope of the present disclosure.

What is claimed is:

1. An electroosmotic pump, comprising a first electrode, a porous membrane, and a second electrode, wherein, the first electrode is close to one side of the porous membrane, and the second electrode is close to another side of the porous membrane;
the porous membrane is a flexible membrane with penetrating pores, or is obtained by modification of the flexible membrane with penetrating pores; and the flexible membrane with penetrating pores is a polycarbonate track-etched membrane with penetrating pores, a polyester track-etched membrane with penetrating pores, a polytetrafluoroethylene track-etched membrane with penetrating pores, or a polyimide track-etched membrane with penetrating pores;
the modification comprises: modifying the flexible membrane with penetrating pores by polydopamine, polyethylene glycol diamine, and bovine serum albumin sequentially;
the first electrode is an anode, and is made of a material comprising a stainless steel mesh, a metal mesh, a metal coating, or a gold-plated stainless steel mesh; and
the second electrode is a cathode, and is made of a material comprising a stainless steel mesh, a metal mesh, a metal coating, or a gold-plated stainless steel mesh.

2. The electroosmotic pump according to claim 1, wherein, the porous membrane has a thickness of 5-200 μm.

3. The electroosmotic pump according to claim 1, wherein, the flexible membrane with penetrating pores is prepared by bombarding a flexible membrane with neutrons, protons, heavy ions, or high-energy particle flows and then conducting chemical etching, wherein the flexible membrane that is bombarded is a polycarbonate membrane, a polyester membrane, a polytetrafluoroethylene membrane, or a polyimide membrane.

4. The electroosmotic pump according to claim 1, wherein, the penetrating pores have a density of 10⁴-10¹⁰ pore/cm² and a pore size of 50-300 nm.

5. The electroosmotic pump according to claim 1, wherein, the modification comprises: soaking the flexible membrane with penetrating pores in a polydopamine solution with a pH of 8.5 for 3-4 h, and rinsing the soaked flexible membrane with deionized water to obtain an intermediate material; and soaking the intermediate material in a polyethylene glycol diamine solution with a pH of 8.5 for 24 h, and transferring the soaked intermediate material to a bovine serum albumin solution with a pH of 7.4 for incubation for 24 h.

6. The electroosmotic pump according to claim 1, wherein, the stainless steel mesh comprises a 304 stainless steel mesh or a 316 stainless steel mesh;
the metal mesh comprises an aluminum mesh, a titanium mesh, or a platinum mesh; and
the metal coating comprises a gold coating or a platinum coating.

7. The electroosmotic pump according to claim 1, wherein, the electroosmotic pump has a thickness of 35 μm to 1.8 mm.

8. An insulin pump, comprising the electroosmotic pump according to claim 7, as a driving pump to drive an automatic injection of insulin.

9. The insulin pump according to claim 8, comprising an insulin storage device, a power supply device, and the electroosmotic pump, wherein,
the insulin storage device is configured to store insulin to be injected; and
the power supply device is configured to power the electroosmotic pump.

10. The insulin pump according to claim 9, wherein, when the porous membrane is the flexible membrane with penetrating pores, the electroosmotic pump has a driving voltage of 0.1-20 V, and insulin has a concentration of 1-50 U/mL; and
when the porous membrane is obtained by the modification of the flexible membrane with penetrating pores, insulin has a concentration of 1-500 U/mL.

11. An insulin pump system, comprising an insulin pump, a sensor device, and an infusion device, wherein, the insulin pump system is configured for automatic pumping of insulin;
the insulin pump is integrated with the sensor device to form a closed-loop system, and the sensor device is configured to measure a glucose concentration, obtain a glucose detection signal, and control opening and closing of an electroosmotic pump according to the detection signal;
the electroosmotic pump is configured to drive insulin to flow to the infusion device;
the infusion device is configured to complete the injection of insulin; and
the insulin pump is the insulin pump according to claim 8.

12. The electroosmotic pump according to claim 2, wherein, the penetrating pores have a density of 10⁴-10¹⁰ pore/cm² and a pore size of 50-300 nm.

13. An insulin pump system, comprising an insulin pump, a sensor device, and an infusion device, wherein, the insulin pump system is configured for automatic pumping of insulin;
the insulin pump is integrated with the sensor device to form a closed-loop system, and the sensor device is configured to measure a glucose concentration, obtain a glucose detection signal, and control opening and closing of an electroosmotic pump according to the detection signal;
the electroosmotic pump is configured to drive insulin to flow to the infusion device;
the infusion device is configured to complete injection of insulin; and
the insulin pump is the insulin pump according to claim 9.

14. An insulin pump system, comprising an insulin pump, a sensor device, and an infusion device, wherein, the insulin pump system is configured for automatic pumping of insulin;
the insulin pump is integrated with the sensor device to form a closed-loop system, and the sensor device is configured to measure a glucose concentration, obtain a glucose detection signal, and control opening and closing of an electroosmotic pump according to the detection signal;
the electroosmotic pump is configured to drive insulin to flow to the infusion device;
the infusion device is configured to complete injection of insulin; and
the insulin pump is the insulin pump according to claim 10.
